(19) United States
(12) Patent Application Publication
(10) Pub. No.: US 2024/0390882 A1
(43) Pub. Date: Nov. 28, 2024

(54) CATIONIC COBALT COMPLEXES FOR ASYMMETRIC HYDROGENATION AND METHODS OF MAKING THE SAME

(71) Applicant: THE TRUSTEES OF PRINCETON UNIVERSITY, Princeton, NJ (US)

(72) Inventors: Paul J. Chirik, Princeton, NJ (US); Connor S. MacNeil, Conshohocken, PA (US)

(21) Appl. No.: 18/719,574

(22) PCT Filed: Dec. 16, 2022
(86) PCT No.: PCT/US2022/053117

§ 371 Date: Jun. 13, 2024

Related U.S. Application Data
(60) Provisional application No. 63/290,157, filed on Dec. 16, 2021.

(51) Int. Cl. B01J 31/24; B01J 31/22; C07F 15/06

(52) U.S. Cl. CPC B01J 31/2452; B01J 31/2295; C07F 15/065; B01J 2231/645; B01J 2531/004; B01J 2531/845

(57) ABSTRACT

In one aspect, methods for the facile synthesis of cationic cobalt complexes for asymmetric hydrogenation of alkenes are provided. Synthetic methods described herein, in some embodiments, provide Co(I) precatalysts on a time scale and yield permitting catalytic evaluation of a significant number of enantiopure ligands.

### RELATED APPLICATION DATA

The present application claims priority pursuant to Article 8 of the Patent Cooperation Treaty to United States Provisional Patent Application Ser. No. 63/290,157 filed Dec. 16, 2021, which is incorporated herein by reference in its entirety.

### FIELD

The present invention relates to transition metal catalysts for asymmetric hydrogenation and, in particular, to cationic Co(I) complexes and methods of making the same.

### BACKGROUND

The asymmetric hydrogenation of olefins is one of the most powerful reactions for the synthesis of single enantiomer products. Traditionally, Schrock-Osborn type catalysts have been employed for asymmetric hydrogenation. While effective, these catalysts carry a high cost, given the rhodium metal center. In view of these economic disadvantages, catalysts employing more abundant transition metals remain elusive.

### SUMMARY

In one aspect, methods for the facile synthesis of cationic cobalt complexes for asymmetric hydrogenation of alkenes are provided. Synthetic methods described herein, in some embodiments, provide cationic Co(I) precatalysts on a time scale and yield permitting catalytic evaluation of a significant number of enantiopure ligands. In some embodiments, a method of making a cationic cobalt (I) complex comprises providing a precursor complex of the formula:

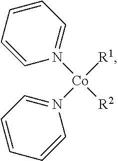

and
substituting ligands of the precursor complex with optically active bis(phosphine) ligand and arene ligand to provide the cationic cobalt (I) complex of formula:

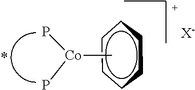

wherein R¹ and R² are independently selected from the group consisting of alkyl and heteroalkyl, and X⁻ is a counterion. As used herein, a heteroalkyl moiety includes one or more heteroatoms in the alkyl chain. In some embodiments, for example, heteroalkyl includes a silicon atom in the alkyl chain. Additionally, in some embodiments, one or both of the pyridine ligands of the precursor complex may be optionally substituted with one or more substituents. Substituents of one or both of the pyridine ligands can comprise any species not inconsistent with the technical objectives described herein. In some embodiments, substituents of the pyridine ligands can enhance crystallinity of the precursor complex, thereby facilitating handling of the precursor complex in synthetic methods described herein. In some embodiments, one or both of the pyridine ligands are independently substituted with alkyl substituents. One or more alkyl substituents may be the same or different between the two pyridine ligands. In some embodiments, the precursor complex is of the formula

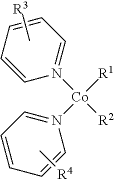

wherein R³ and R⁴ are independently alkyl. R³ and R⁴ can be the same or different.

Additionally, in some embodiments, various silver salts can be employed in oxidation of bis(phosphine) cobalt dialkyl complexes in the synthesis of cationic cobalt complexes described herein. In some embodiments, for example, silver salts of AgOTf, AgSbF₆ and/or AgBF₄ can replace ferrocenium salts in the oxidation.

Moreover, in some embodiments, the optically active bis(phosphine) ligand can displace the pyridine ligands of the precursor complex to provide an intermediate complex of the formula:

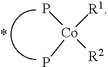

Additionally, R¹ and R² can be any alkyl or heteroalkyl consistent with displacement by arene ligand in the formation of the cationic Co(I) complex. In some embodiments, for example, at least one of R¹ and R² is —CH₂SiMe₃.

In another aspect, a method of making a cationic cobalt (I) complex comprises providing a cationic cobalt (I) arene sandwich precursor complex, and substituting an arene ligand of the precursor complex with an optically active bis(phosphine) ligand to provide the cationic cobalt (I) complex of formula:

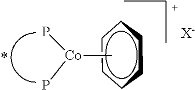

wherein X⁻ is a counterion. In some embodiments, the cationic cobalt (I) arene sandwich precursor complex is of the formula:

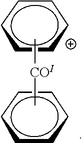

In another aspect, methods of asymmetric alkene hydrogenation are described herein. In some embodiments, a method of asymmetric alkene hydrogenation comprises providing an alkene substrate, and hydrogenating the alkene in the presence of a cationic cobalt (I) catalyst to yield a single enantiomer reaction product, wherein the cationic cobalt (I) catalyst is derived from a precatalyst of the formula:

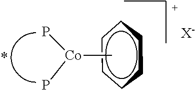

wherein

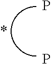

is optically active bis(phosphine) ligand, and X⁻ is a counterion. The precatalyst is synthesized by providing a precursor complex of the formula:

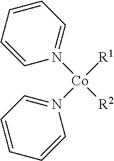

wherein one or both of the pyridine ligands are optionally substituted with one or more alkyl substituents, and substituting ligands of the precursor complex with the optically active bis(phosphine) ligand and arene ligand, wherein R¹ and R² are independently selected from the group consisting of alkyl and heteroalkyl. In some embodiments, the alkene substrate is a pharmaceutical compound or a pharmaceutical precursor.

In another aspect, a method of asymmetric alkene hydrogenation comprises providing an alkene substrate, and hydrogenating the alkene in the presence of a cationic cobalt (I) catalyst to yield a single enantiomer reaction product, wherein the cationic cobalt (I) catalyst is derived from a precatalyst of the formula:

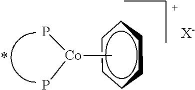

wherein

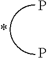

is optically active bis(phosphine) ligand, and X⁻ is a counterion. The precatalyst is synthesized by providing a cationic cobalt (I) arene sandwich precursor complex, and substituting an arene ligand of the precursor complex with the optically active bis(phosphine) ligand.

In methods described herein, the optically active bis(phosphine) ligand can be enantiopure. In some embodiments, the optically active bis(phosphine) ligand is selected from the group consisting of DuPhos, BenzP*, TangPhos, and DuanPhos. Moreover, X can be any counterion consistent with the technical objectives of the present application.

Synthetic methods described herein can yield cationic Co(I) complexes in an amount of at least 90% or 95%, in some embodiments. Additionally, reaction time for producing the cationic Co(I) complexes according to synthetic methods described herein can be less than 10 minutes. In some embodiments, reaction time for Co(I) catalyst production ranges from 1-10 minutes. Such high yields and fast reaction times permit catalytic evaluation of a significant number of enantiopure ligands when incorporated into the Co(I) architecture.

These and other embodiments are further described in the following detailed description.

### BRIEF DESCRIPTION OF THE DRAWINGS

FIG. 1 illustrates various cationic Co(I) complexes synthesized according to methods described herein.

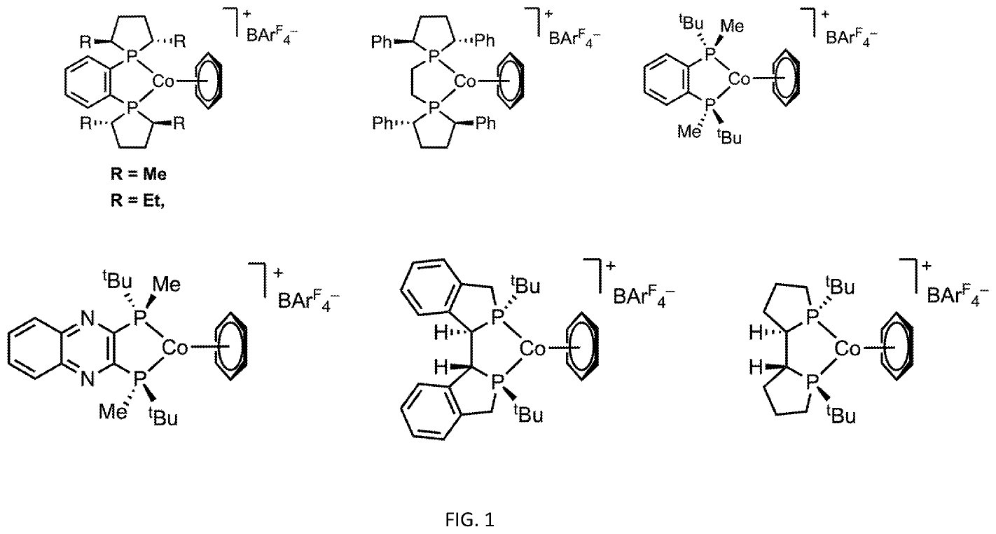

FIG. 2 illustrates various alkene substrates for asymmetric hydrogenation with Co(I) complexes described herein.

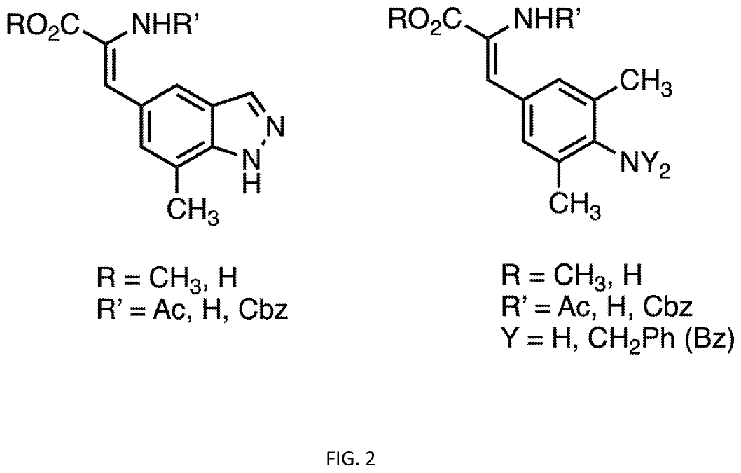

FIG. 3 illustrates a synthetic route for a Co(I) complex according to one embodiment.

FIG. 4 illustrates a synthetic route for a Co(I) complex according to one embodiment.

FIG. 5 illustrates a synthetic route for a Co(I) complex according to one embodiment.

FIG. 6 illustrates a synthetic route for a Co(I) complex according to one embodiment.

FIG. 7 illustrates a synthetic route for a Co(I) complex according to one embodiment.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Example 1—Synthesis of Cationic Co(I) Complex for Asymmetric Hydrogenation

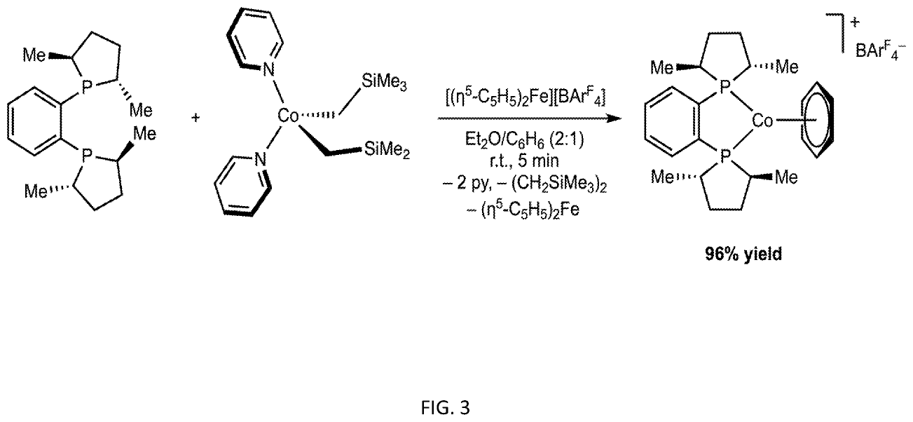

A Co(I) complex for asymmetric hydrogenation was prepared according to the reaction scheme of FIG. 3 and as follows.

Preparation of [(S,S)-(^{Me}DuPhos) Co(η⁶—C₆H₆)][BAr^{F}₄]. In a nitrogen-filled glovebox, a 20 mL scintillation vial was charged with (py)₂Co(CH₂SiMe₃)₂ (0.097 g, 0.24 mmol) as a dark green semi-solid and 2 mL of diethyl ether were added. In a separate vial, (S,S)-^{Me}DuPhos (0.078 g, 0.25 mmol) was weighed out as a crystalline white solid and dissolved in diethyl ether. To the ethereal solution of (S,S)-^{Me}DuPhos was added (py)₂Co(CH₂SiMe₃)₂ dropwise. The dark green ethereal solution of (py)₂Co(CH₂SiMe₃)₂ instantly turned dark red when combined with bis(phosphine) and was stirred for 30 seconds at ambient temperature. Single-crystals of (S,S)-(^{Me}DuPhos) Co(CH₂SiMe₃)₂ deposited on the sides of the scintillation vial could be collected and used in the oxidatively-induced reductive elimination, however, routine precatalyst synthesis was done in one-pot. Finally, [(η⁵—C₅H₅)₂Fe][BAr^{F}₄] (0.251 g, 0.24 mmol) was weighed out as a deep blue solid and dissolved in 3 mL of a mixture of diethyl ether and benzene (2:1). When [(η⁵—C₅H₅)₂Fe][BAr^{F}₄] was added dropwise to the dark red ethereal solution of (S,S)-(^{Me}DuPhos) Co(CH₂SiMe₃)₂ (formed in situ), the deep blue color instantly gave way to bright yellow. The reaction mixture was stirred for 5 minutes at ambient temperature, at which point the solution became cloudy. Volatiles were removed under reduced pressure and the residue was washed with pentane to remove ferrocene and silane byproducts. The residue was taken up in diethyl ether and passed through a pad of Celite where it was then dried under reduced pressure to yield 0.301 g (96% yield) of [(S,S)-(^{Me}DuPhos)Co(η⁶—C₆H₆)][BAr^{F}₄] as an orange crystalline powder.

Anal Calcd for C₅₂H₄₆BCoF₂₄P₂: C, 51.48; H, 3.55. Found: C, 51.5; H, 3.2. ¹H NMR (500 MHz, THF-d₈, 23° C.): δ 7.79 (br m, 8H, o-B[(3,5-(CF₃)₂)C₆H₃]₄), 7.68 (br m, 2H, ^{iPr}DuPhos Ar—H), 7.59 (br m, 2H, ^{iPr}DuPhos Ar—H), 7.57 (br m, 4H, p-B[(3,5-(CF₃)₂)C₆H₃]₄), 6.38 (s, 6H, η⁶—C₆H₆), 2.65 (br m, 2H, ^{iPr}DuPhos CH), 2.34 (br m, 2H, ^{iPr}DuPhos CH), 2.31 (br m, 2H, ^{iPr}DuPhos CH₂), 2.20 (br m, 2H, ^{iPr}DuPhos CH₂), 1.80 (br m, 2H, ^{iPr}DuPhos CH₂), 1.55 (br m, 2H, ^{iPr}DuPhos CH₂), 1.37 (br m, 6H, ^{iPr}DuPhos CH₃), 0.86 (br m, 6H, ^{iPr}DuPhos CH₃). ¹³C{¹H} NMR (125.7 MHz, THF-d₈, 23° C.): δ 162.9 (q, ¹J_{B-C}=50.1 Hz, B[(3,5-(CF₃)₂)C₆H₃]₄), 143.8 (app t, ^{iPr}DuPhos Ar), 135.8 (br s, B[(3,5-(CF₃)₂)C₆H₃]₄), 132.5 (s, ^{iPr}DuPhos Ar), 132.0 (app t, ^{iPr}DuPhos Ar), 130.2 (m, B[(3,5-(CF₃)₂)C₆H₃]₄), 125.7 (q, ¹J_{C-F}=272.5 Hz, B[(3,5-(CF₃)₂)C₆H₃]₄), 118.4 (br s, B[(3,5-(CF₃)₂)C₆H₃]₄), 93.6 (s, η⁶—C₆H₆), 45.9 (app t, ^{iPr}DuPhos CH), 42.6 (app t, ^{iPr}DuPhos CH), 37.2 (^{iPr}DuPhos CH₂), 36.8 (^{iPr}DuPhos CH₂), 17.9 (^{iPr}DuPhos CH₃), 13.9 (^{iPr}DuPhos CH₃). ³¹P{¹H} NMR (202.4 MHz, THF-d₈, 23° C.): δ 96.2 (s, 2P). ¹⁹F NMR (376.5 MHz, THF-d₈, 23° C.): δ −63.4 (s, B[(3,5-(CF₃)₂)C₆H₃]₄).

### Example 2—Synthesis of Cationic Co(I) Complex for Asymmetric Hydrogenation

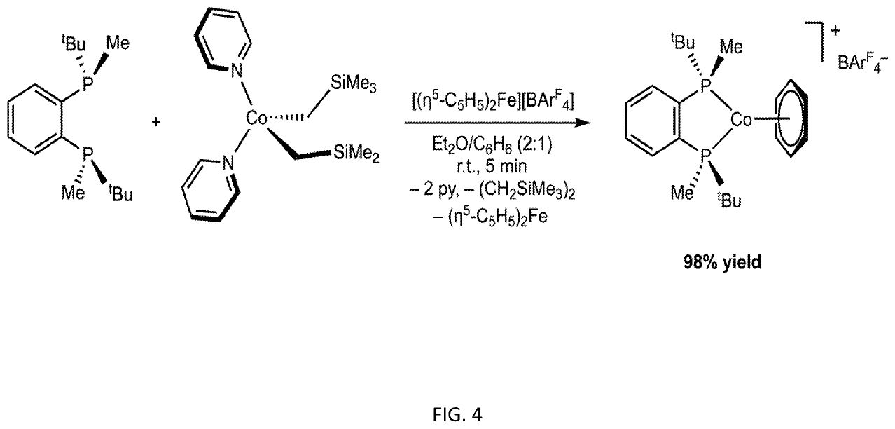

A Co(I) complex for asymmetric hydrogenation was prepared according to the reaction scheme of FIG. 4 and as follows.

Preparation of [(R,R)-(BenzP*)Co(η⁶—C₆H₆)][BAr^{F}₄]. In a nitrogen-filled glovebox, a 20 mL scintillation vial was charged with (py)₂Co(CH₂SiMe₃)₂ (0.051 g, 0.13 mmol) as a dark green semi-solid and 2 mL of diethyl ether were added. In a separate vial, (R,R)-BenzP* (0.037 g, 0.13 mmol) was weighed out as a crystalline white solid and dissolved in diethyl ether. To the diethyl ether solution of (R,R)-BenzP* was added a diethyl ether solution of (py)₂Co(CH₂SiMe₃)₂ dropwise. A color change to bright orange was observed immediately and the mixture was stirred for an additional 30 seconds at ambient temperature. Finally, [(η⁵—C₅H₅)₂Fe][BAr^{F}₄] (0.136 g, 0.13 mmol) was weighed out as a deep blue solid and dissolved in a mixture of diethyl ether and benzene (2:1). When [(η⁵—C₅H₅)₂Fe][BAr^{F}₄] was added dropwise to the stirring ethereal solution of (R,R)-(BenzP*)Co(CH₂SiMe₃)₂ (formed in situ), the deep blue color instantly gave way to bright yellow. The reaction mixture was stirred for 5 minutes at ambient temperature, at which point the solution became cloudy. Volatiles were removed under reduced pressure and the residue was washed with pentane to remove ferrocene and silane byproducts. The residue was extracted into diethyl ether and passed through a pad of Celite where it was then dried under reduced pressure to yield 0.163 g (99% yield) of [(R,R)-(BenzP*)Co(η⁶—C₆H₆)][BAr^{F}₄] as an orange crystalline powder. Anal Calcd for C₅₄H₄₆BCoF₂₄P₂: C, 50.57; H, 3.62. Found: C, 46.07; H, 2.75. ¹H NMR (500 MHz, THF-d₈, 23° C.): δ ¹H NMR (500 MHz, THF-d₈, 23° C.): δ 7.79 (br m, 8H, o-B[(3,5-(CF₃)₂)C₆H₃]₄), 7.76 (br m, 2H, BenzP* Ar), 7.64 (br m, 2H, BenzP* Ar), 7.57 (br m, p-B[(3,5-(CF₃)₂)C₆H₃]₄), 6.38 (s, 6H, η⁶—C₆H₆), 1.89 (m, 6H, BenzP* CH₃), 0.93 (m, 18H, BenzP* CH₃). ¹³C{¹H} NMR (125.7 MHz, THF-d₈, 23° C.): δ 162.9 (q, ¹J_{B-C}=51.4 Hz, B[(3,5-(CF₃)₂)C₆H₃]₄), 135.7 (br s, B[(3,5-(CF₃)₂)C₆H₃]₄), 131.9 (s, BenzP* Ar), 131.4 (app t, BenzP* Ar), 127.9 (q, ¹J_{C-F}=269.9 Hz, B[(3,5-(CF₃)₂)C₆H₃]₄), 118.3 (br m, B[(3,5-(CF₃)₂)C₆H₃]₄), 93.3 (s, η⁶—C₆H₆), 36.8 (m, BenzP* CH₃), 27.4 (s, BenzP* C(CH₃)₃), 11.7 (s, BenzP* C(CH₃)₂). ³¹P{¹H} NMR (202.4 MHz, THF-d₈, 23° C.): δ 77.3 (s, 2P). ¹⁹F NMR (376.5 MHz, THF-d₈, 23° C.): 6-63.4 (s, B[(3,5-(CF₃)₂)C₆H₃]₄).

### Example 3—Synthesis of Cationic Co(I) Complex for Asymmetric Hydrogenation

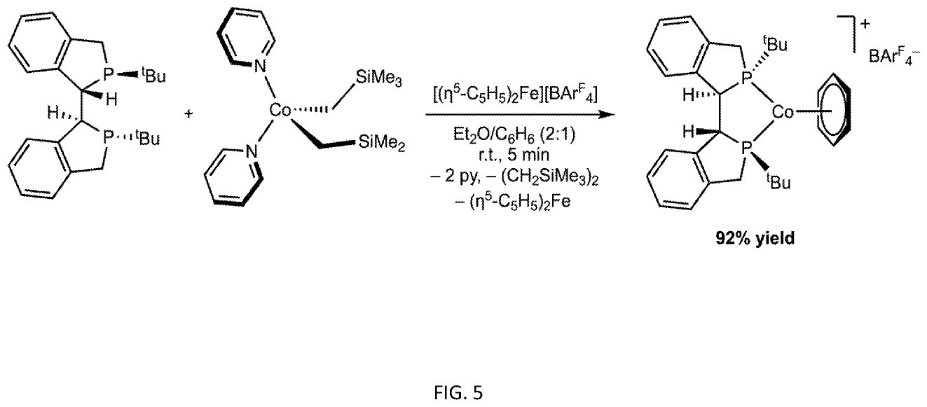

A Co(I) complex for asymmetric hydrogenation was prepared according to the reaction scheme of FIG. 5 and as follows.

Preparation of [(1S,1′S,1R,1′R)-(DuanPhos)Co(η⁶—C₆H₆)][BAr^{F}₄]. In a nitrogen-filled glovebox, a 20 mL scintillation vial was charged with (py)₂Co(CH₂SiMe₃)₂ (0.070 g, 0.18 mmol) as a dark green semi-solid and 2 mL of diethyl ether were added. In a separate vial, (1S,1′S,1R,1′R)-DuanPhos (0.075 g, 0.18 mmol) was weighed out as a crystalline orange solid and dissolved in diethyl ether. To the ethereal solution of (1S,1′S,R,1′R)-DuanPhos was added a diethyl ether solution of (py)₂Co(CH₂SiMe₃)₂ dropwise. A color change to bright orange was observed and the reaction mixture was stirred an additional 30 seconds at ambient temperature.

Finally, [(η⁵—C₅H₅)₂Fe][BAr^{F}₄] (0.188 g, 0.18 mmol) was weighed out as a deep blue solid and dissolved in a mixture of diethyl ether and benzene (2:1). The solution containing the [(η⁵—C₅H₅)₂Fe][BAr^{F}₄] was added dropwise to the stirring ethereal solution of (1S,1′S,R,1′R)-(DuanPhos)Co(CH₂SiMe₃)₂ (formed in situ), and a color change from deep blue to dark orange was observed. The reaction mixture was stirred for 5 minutes at ambient temperature, at which point the solution became cloudy. Volatiles were removed under reduced pressure and the residue was washed with pentane to remove ferrocene and silane byproducts. The residue was taken up in diethyl ether and passed through a pad of Celite where it was then dried under reduced pressure to yield 0.253 g (92% yield) of [(1S,1′S,1R,1′R)-(DuanPhos)Co(η⁶—C₆H₆)][BAr^{F}₄] as a dark orange crystalline powder. Anal Calcd for C₆₂H₅₀BCoF₂₄P₂: C, 50.41; H, 3.92. Found: C, 50.04; H, 3.51. ¹H NMR (500 MHz, THF-d₈, 23° C.): at 23° C., spectra were broad and uninformative. ³¹P{¹H} NMR (202.4 MHz, THF-d₈, 23° C.): δ 127.2 (s, 2P). ¹⁹F NMR (376.5 MHz, THF-d₈, 23° C.): δ −63.5 (s, B[(3,5-(CF₃)₂)C₆H₃]₄).

### Example 4—Synthesis of Cationic Co(I) Complex for Asymmetric Hydrogenation

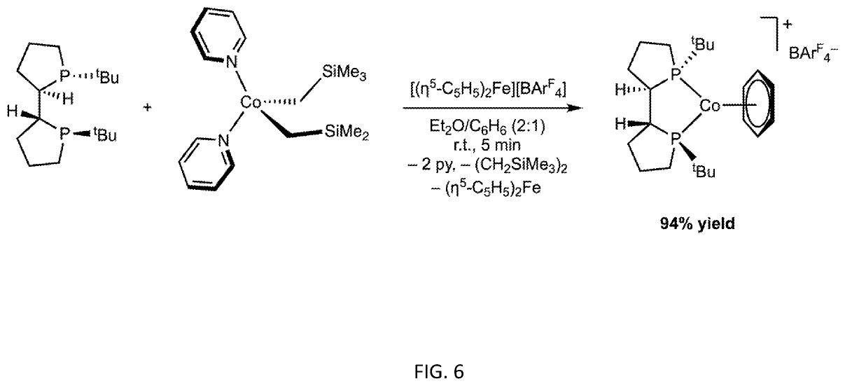

A Co(I) complex for asymmetric hydrogenation was prepared according to the reaction scheme of FIG. 6 and as follows.

Preparation of [(S,S′,R,R′)-(TangPhos)Co(η⁶—C₆H₆)][BAr^{F}₄]. In a nitrogen-filled glovebox, a 20 mL scintillation vial was charged with (py)₂Co(CH₂SiMe₃)₂ (0.070 g, 0.18 mmol) as a dark green semi-solid and 2 mL of diethyl ether were added. In a separate vial, (S,S′,R,R′)-TangPhos (0.075 g, 0.18 mmol) was weighed out as a crystalline orange solid and dissolved in diethyl ether. To the ethereal solution of (S,S′,R,R′)-TangPhos was added (py)₂Co(CH₂SiMe₃)₂ dropwise. The dark green ethereal solution of (py)₂Co(CH₂SiMe₃)₂ instantly turned bright orange when combined with bis(phosphine) and was stirred for 30 seconds at ambient temperature.

Finally, [(η-C₅H₅)₂Fe][BAr^{F}₄] (0.188 g, 0.18 mmol) was weighed out as a deep blue solid and dissolved in a mixture of diethyl ether and benzene (2:1). When [(η⁵—C₅H₅)₂Fe][BAr^{F}₄] was added dropwise to the stirring ethereal solution of (S,S′,R,R′)-(TangPhos)Co(CH₂SiMe₃)₂ (formed in situ), the deep blue color instantly gave way to dark orange. The reaction mixture was stirred for 5 minutes at ambient temperature, at which point the solution became cloudy. Volatiles were removed under reduced pressure and the residue was washed with pentane to remove ferrocene and silane byproducts. The residue was taken up in diethyl ether and passed through a pad of Celite where it was then dried under reduced pressure to yield 0.253 g (92% yield) of [(S,S′,R,R′)-(TangPhos)Co(η⁶—C₆H₆)][BAr^{F}₄] as a dark orange crystalline powder. Anal Calcd for C₅₄H₅₀BCoF₂₄P₂: C, 50.41; H, 3.92. Found: C, 50.04; H, 3.51. ¹H NMR (500 MHz, THF-d₈, 23° C.): δ 7.78 (br m, 8H, o-B[(3,5-(CF₃)₂)C₆H₃]₄), 7.57 (br m, p-B[(3,5-(CF₃)₂)C₆H₃]₄), 6.26 (s, 6H, η⁶—C₆H₆), 2.43-2.31 (ov m, 4H, TangPhos CH+TangPhos CH₂), 2.09-1.94 (ov m, 6H, TangPhos CH₂), 1.63 (m, 2H, TangPhos CH₂), 1.17 (m, 18H, TangPhos C(CH₃)₃). ¹³C{¹H} NMR (125.7 MHz, THF-d₈, 23° C.): δ 162.9 (q, ¹J_{B-C}=50.1 Hz, B[(3,5-(CF₃)₂)C₆H₃]₄), 135.7 (br s, B[(3,5-(CF₃)₂)C₆H₃]₄), 130.1 (br m, B[(3,5-(CF₃)₂)C₆H₃]₄), 125.7 (q, ¹J_{C-F}=272.2 Hz, B[(3,5-(CF₃)₂)C₆H₃]₄), 118.3 (br m, B[(3,5-(CF₃)₂)C₆H₃]₄), 92.8 (s, η⁶—C₆H₆), 46.5 (app t, TangPhos CH), 37.4 (app t, TangPhos CH₂), 35.5 (app t, TangPhos CH₂), 33.6 (app t, TangPhos CH₂), 28.5 (s, TangPhos C(CH₃)₃), 28.2 (s, TangPhos C(CH₃)₃). ³¹P{¹H} NMR (202.4 MHz, THF-d₈, 23° C.): δ 121.7 (s, 2P). ¹⁹F NMR (376.5 MHz, THF-d₈, 23° C.): 6-63.4 (s, B[(3,5-(CF₃)₂)C₆H₃]₄).

### Example 5—Synthesis of Cationic Co(I) Complex for Asymmetric Hydrogenation

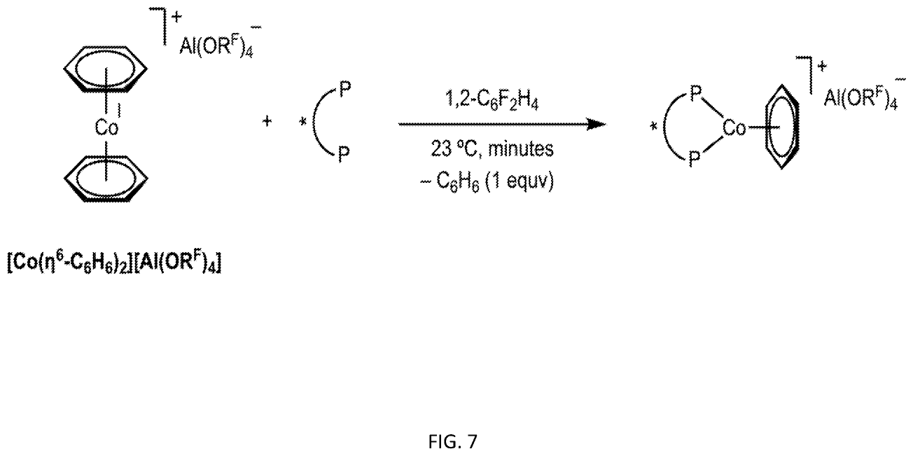

A Co(I) complex for asymmetric hydrogenation was prepared according to the reaction scheme of FIG. 7 and as follows.

This method utilizes a 20-electron arene ‘sandwich’ cation, (e.g. [Co(η⁶—C₆H₆)₂][X]) stabilized by either the weakly coordinating anion (WCA), [Al(OR^{F})₄]⁻ (R^{F}═C(CF₃)₃) developed by the group of Ingo Krossing, or commonly-used anions including BAr^{F}₄⁻, SbF₆⁻, PF₆⁻, and BF₄⁻. We have shown that combinations of [Co(η⁶—C₆H₆)₂][Al(OR^{F})₄] and optically-active bis(phosphines) including (R,R)-^{iPr}DuPhos, lead to air-stable, 18-electron bis(phosphine) cobalt cations similar to those described above with the key distinction being the composition of the anion. The reaction to generate the 18-electron cations must be carried out in 1,2-difluorobenzene given the poor stability of the 20-electron arene ‘sandwich’ complex [Co(η⁶—C₆H₆)₂][Al(OR^{F})₄] in coordinating solvents including THF. A representative example is given below: In a nitrogen-filled glovebox, a 20 mL scintillation vial was charged with [Co(η⁶—C₆H₆)₂][Al(OR^{F})₄] (0.010 g, 8.5 μmol) and dissolved in 2 mL of 1,2-difluorobenzene to give an amber-colored solution. Separately, (R,R)-^{iPr}DuPhos (0.004 g, 8.6 μmol) was dissolved in 2 mL of benzene and added to [Co(η⁶—C₆H₆)₂][Al(OR^{F})₄] at ambient temperature resulting in an immediate color change from amber to bright yellow. Characterization of the product by multinuclear NMR spectroscopy in THF-d₈ gave nearly-identical shifts to the 18-electron cobalt complex [(R,R)-(^{iPr}DuPhos)Co(η⁶—C₆H₆)][BAr^{F}₄] with the exception of the ¹⁹F NMR spectrum which contained a single peak corresponding to the CF₃ groups of the aluminate anion. The activity of this complex was surveyed in the asymmetric hydrogenation of dehydro-sitagliptin where it maintained excellent enantioselectivity (96% ee) at 50° C. under 1000 psi of H₂.

### Example 6—Synthesis of Cationic Co(I) Complex for Asymmetric Hydrogenation

Preparation of [(RR)-(^{iPr}DuPhos)Co(η⁶—C₆H₆)][BAr^{F}₄]. In a nitrogen-filled glovebox, a 20 mL scintillation vial was charged with (3,5-(Me)₂-C₅H₃N)₂Co(CH₂SiMe₃)₂ (0.081 g, 0.18 mmol) as a dark green crystalline solid and dissolved in 2 mL of diethyl ether. In a separate vial, (R,R)-^{iPr}DuPhos (0.075 g, 0.18 mmol) was weighed out as a crystalline white solid and dissolved in diethyl ether. To the ethereal solution of (R,R)-^{iPr}DuPhos was added (3,5-(Me)₂-C₅H₃N)₂Co(CH₂SiMe₃)₂ dropwise. The dark green ethereal solution of (3,5-(Me)₂-C₅H₃N)₂Co(CH₂SiMe₃)₂ instantly turned bright orange when combined with bis(phosphine) and was stirred for 30 seconds at ambient temperature. Finally, [(η⁵—C₅H₅)₂Fe][BAr^{F}₄] (0.188 g, 0.18 mmol) was weighed out as a deep blue solid and dissolved in a mixture of diethyl ether and benzene (2:1). When [(η⁵—C₅H₅)₂Fe][BAr^{F}₄] was added dropwise to the stirring ethereal solution of (R,R)-(^{iPr}DuPhos)Co(CH₂SiMe₃)₂ (formed in situ), the deep blue color instantly gave way to bright yellow. The reaction mixture was stirred for 5 minutes at ambient temperature, at which point the solution became cloudy. Volatiles were removed under reduced pressure and the residue was washed with pentane to remove ferrocene and silane byproducts. The residue was taken up in diethyl ether and passed through a pad of Celite where it was then dried under reduced pressure to yield 0.251 g (98% yield) of [(R,R)-(^{iPr}DuPhos)Co(η⁶—C₆H₆)][BAr^{F}₄] as an orange-yellow crystalline powder. Characterization data were consistent with our previously-reported method involving chloride abstraction from [(R,R)-(^{iPr}DuPhos)Co(μ-Cl)]₂ with NaBAr^{F}₄.

Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A method of making a cationic cobalt(I) complex comprising:
providing a precursor complex of the formula: 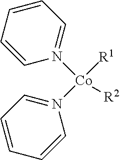
wherein one or both of the pyridine ligands are optionally substituted with one or more substituents; and
substituting ligands of the precursor complex with optically active bis(phosphine) ligand and arene ligand to provide the cationic cobalt(I) complex of formula: 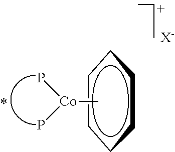
wherein R¹ and R² are independently selected from the group consisting of alkyl and heteroalkyl, and X⁻ is a counterion.

2. The method of claim 1, wherein the optically active bis(phosphine) ligand is enantiopure.

3. The method of claim 1, wherein the optically active bis(phosphine) ligand displaces the pyridine ligands of the precursor complex to provide an intermediate complex of the formula:

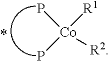

4. The method of claim 1 having a yield of the cationic cobalt(I) complex of greater than 90%.

5. The method of claim 1, wherein the cationic cobalt(I) complex is synthesized in a time period less than 10 minutes.

6. The method of claim 1, wherein the cationic cobalt(I) complex is synthesized in a time period of 5 minutes of less.

7. The method of claim 1, wherein the optically active bis(phosphine) ligand is selected from the group consisting of DuPhos, BenzP*, TangPhos, and DuanPhos.

8. The method of claim 1, wherein the precursor complex is of the formula 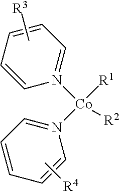
wherein R³ and R⁴ are independently alkyl.

9. A method of asymmetric alkene hydrogenation comprising:
providing an alkene substrate, and hydrogenating the alkene substrate in the presence of a cationic cobalt(I) catalyst to yield a single enantiomer reaction product, wherein the cationic cobalt(I) catalyst is derived from a precatalyst of the formula: 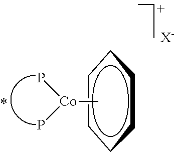
wherein 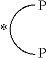
is optically active bis(phosphine) ligand, and X⁻ is a counterion, the precatalyst being synthesized by providing a precursor complex of the formula: 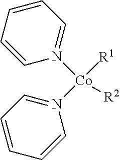
wherein one or both of the pyridine ligands are optionally substituted with one or more substituents, and
substituting ligands of the precursor complex with the optically active bis(phosphine) ligand and arene ligand, wherein R¹ and R² are independently selected from the group consisting of alkyl and heteroalkyl.

10. The method of claim 9, wherein the optically active bis(phosphine) ligand is selected from the group consisting of DuPhos, BenzP*, TangPhos, and DuanPhos.

11. The method of claim 9, wherein the alkene substrate is a pharmaceutical compound or a pharmaceutical precursor.

12. The method of claim 11, wherein the alkene substrate is of the formula: 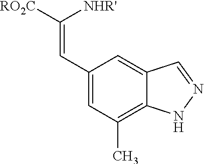
wherein R is selected from the group consisting of hydrogen and alkyl, and R′ is selected from the group consisting of hydrogen, acetate, and benzyl chloroformate.

13. The method of claim 11, wherein the alkene is of the formula: 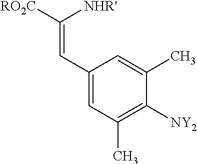
is selected from the group consisting of hydrogen and alkyl, and R′ is selected from the group consisting of hydrogen, acetate, and benzyl chloroformate, and Y is selected from the group consisting of hydrogen and aryl.

14. The method of claim 9, wherein the precursor complex is of the formula 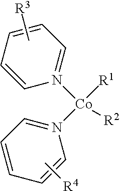
wherein R³ and R⁴ are independently alkyl.

15. A transition metal complex of the formula: 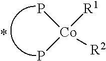
wherein 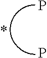
is optically active bis(phosphine) ligand, and R¹ and R² are independently selected from the group consisting of alkyl and heteroalkyl.

16. The transition metal complex of claim 15, wherein the optically active bis(phosphine) ligand is selected from the group consisting of DuPhos, BenzP*, TangPhos, and DuanPhos.

17. A method of making a cationic cobalt (I) complex comprising:
providing a cationic cobalt(I) arene sandwich precursor complex, and substituting an arene ligand of the precursor complex with an optically active bis(phosphine) ligand to provide the cationic cobalt(I) complex of formula: 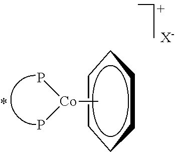
wherein X⁻ is a counterion.

18. The method of claim 17, wherein the cationic cobalt(I) arene sandwich precursor complex is of the formula: [chemical structure]
